(19) United States
(12) Patent Application Publication
(10) Pub. No.: US 2005/0079210 A1
(43) Pub. Date: Apr. 14, 2005

(54) Liposomal delivery system for topical pharmaceutical, cosmeceutical, and cosmetic ingredients

(76) Inventor: Shyam K. Gupta, Scottsdale, AZ (US)

Correspondence Address: TOD R. NISSLE, P.C., P.O. Box 55630, Phoenix, AZ, 85078, US

(21) Appl. No.: 10/681,938

(22) Filed: Oct. 9, 2003

(51) Int. Cl. A61K 9/127

(52) U.S. Cl. 424/450

(57) ABSTRACT

A method of producing a lipophilic liposomal composition includes the step of forming a liposome forming composition by admixing at an elevated temperature at least one lipophilic ingredient selected from a group consisting of pharmaceutical lipophilic ingredients, cosmetic lipophilic ingredients, and cosmeceutical lipophilic ingredients; a liposome-forming lipid component; and, a solubliizer for the liposome-forming lipid component. The resulting liposome forming composition is then cooled.

This invention pertains to systems for delivering topical pharmaceutical, cosmeceutical, and cosmetic ingredients.

More particularly, the invention pertains to a system for forming liposomal pharmaceutical—cosmeceutical carriers by cooling a polyamide—solubilizer—lipophilic—cosmetic/pharmaceutical composition.

In a further respect, the invention pertains to a system for forming a liposomal carrier by combining a polyamide and solubilizer at an elevated temperature, by cooling the resulting composition, and by adding a cosmetic—pharmaceutical ingredient to cause the spontaneous production of liposomes.

The use of liposomes as delivery systems for cosmetics and pharmaceuticals has been known for some time.

A liposome is also defined as a hollow spherical colloidal structure consisting of one or more concentric phospholipid bilayers separated by water or aqueous buffer compartments. Hollow liposome structures also have an internal aqueous compartment. Liposomes can be prepared in diameters ranging from 20 nm (nanometers) to 10 microns, and are classified according to their final size and preparation method. For example, SUV liposomes are small unilamellar vesicles (0.5-50 nm). LUV liposomes are large unilamellar vesicles (100 nm). REV liposomes are reverse phase evaporation vesicles (0.5 micrometer). MLV liposomes are large mutlilamellar vesicles (21-10 micrometers). Drug molecules can either be encapsulated in the enclosed aqueous space or be intercalated into the lipid bilayer.

The exact location of a cosmetic or drug ingredient in a liposome depends on its physicochemical characteristics and the composition of the lipids in the liposome.

Liposomes are effective for sustained release of drug or cosmetic ingredients, but have the drawback that the amount of such ingredients that can be contained in the liposome is limited. One difficulty encountered in the preparation of such sustained release liposomes is producing liposomes that are stable. Another difficulty is producing liposomes that have a high concentration of a desired ingredient. A further difficulty is that all types of unilamellar vesicles and single bilayer liposomes have a relatively low content of lipid molecules and, consequently, have a low loading capacity for lipophilic compounds. Such liposomes are more suitable for the entrapment of water-soluble materials. Multilamellar liposomes can be used to encapsulate large amount of hydrophobic ingredients, but are not appropriate for many topical or pharmaceutical applications because their large size prevents absorption into the skin and they tend to be unstable.

Various methods have been reported for preparing conventional liposomes and for using liposomes to apply topically pharmaceutical, cosmetic, and cosmeceutical ingredients. Drawbacks of conventional liposomes include their instability during storage, low reproducibility during manufacture, low entrapment efficiency, and leakage of loaded ingredients from the liposomes.

Unconventional liposomes sometimes remedy the drawbacks of conventional liposomes. One two-phase process makes liposomes in which one phase is a water-immiscible organic solvent and the other phase is an aqueous phase. This manufacturing process requires, however, a complex balance of solvent ratio, mixing speed, temperature, and an inert gas flow that are mathematically calculated. The process is impractical in normal production environments.

Another unconventional liposome is a combination of a soluble polysaccharide and lipophilic material incorporated in spherical beads that are loaded with skin beneficial ingredients. The preparation of the spherical beads still requires, however, two processing steps, which limits commercial applications of the beads.

A further unconventional liposome is prepared from amphiphilic materials. The manufacturing process for amphiphilic liposomes is complex and of limited scope.

Other liposomes include cationic lipid compounds. Such have been conjugated in a variety of moieties including, for example, carboxyspermine that is conjugated to one of two types of lipids and includes compounds such as 5-carboxyspermylglycine dioctyloleoylamide (“DOGS”) and dipalmitoyl-phosphatidylenthanolamine 5-carboxyspermylamide (“DPPES”). Of the cationic lipids that have been proposed for use in delivering biomolecules to cells, no particular cationic lipid has been reported to work well with a wide variety of ingredients. Since cell types differ from one another in membrane composition, different cationic lipid compositions and different types of lipid aggregates may be effective for different cell types, either due to their ability to contact and fuse with target cell membranes directly or due to different interactions with intracellular membranes or the intracellular environment. For these and other reasons, design of effective lipids, including cationic lipids, has largely been empirical. I addition to content and transfer, other factors believed important include, for example, ability to form lipid aggregates suited to the intended purpose, toxicity of the composition to the target cell, stability as a carrier for the biomolecule to be delivered, and function in an in vivo environment.

Therefore, there is a need for improved liposomes that are capable of delivering on skin surfaces with greater absorption and bioavailability cosmetic and pharmaceutical ingredients to a wide variety of cell types.

It would be highly desirable to develop an unconventional liposome system (1) in which liposomes are made in a single-phase system, (2) in which the liposomes can be loaded with relatively large amounts (5% to 50% for example) of lipophilic cosmetic and pharmaceutical ingredients, (3) using a non-aqueous system; (4) using a simple manufacturing process, (5) to deliver a wide variety of various lipophilic ingredients, (6) in which the size of liposome particles is not critical, and (7) using liposomes that can change their size by the application of mechanical force to facilitate the application of liposomes to skin or to facilitate delivering the liposomes from a dispensing system that has a narrow orifice.

I have discovered a surprisingly simple preparation of an acyl amino acid amide based liposome that is electrically neutral and that is therefore useful for the topical delivery of a wide variety of lipophilic ingredients. The lipophilic ingredients can be electrically positive, negative, or neutral; are not limited to any specific chemical structural moieties or cellular surfaces on skin; and, can be loaded in the liposomes in large concentrations in, for example, the range of 5% to 50% by weight. Other features of the invention are that the liposomal formulations can contain high levels, such as up to 80% by weight, of a vegetable oil as a carrier or solubilizer ingredient; the liposomal formulations do not feel oily on skin surface; and, the liposomal formulations are rapidly absorbed into the skin. It is theorized that the amide-based liposomes carry a lipophilic “tail” that is believed to help the rapid absorption of oily and lipophilic ingredients used in the system of the invention.

I have also discovered a liposome and method of forming the same by forming a liposomal composition by combining at an elevated temperature a polyamide plus a solubilizer plus a lipophilic cosmetic/pharmaceutical ingredient, and then by cooling the composition to cause liposomes to form. The elevated temperature is in the range of 30 degrees C. to 150 degrees C., preferably 70 degrees C. to 120 degrees C.

I have also discovered a liposome and method of forming the same by forming a composition by combining a polyamide plus a solubilizer at an elevated temperature, by cooling the composition, and by addiing a lipophilic cosmetic/pharmaceutical ingredient to cause the spontaneous formation of the liposome. The elevated temperature is in the range of 30 degrees C. to 150 degrees C., preferably 70 degrees C. to 120 degrees C.

As used herein, the terms listed below have the meanings set forth therewith.
Cosmeceutical ingredient. A cosmetic ingredient that may have the efficacy of a pharmaceutical ingredient.
Cosmetic ingredient. An ingredient that is used in the composition of a cosmetic product and that has no permanent or long-term biological benefits.
Disease. An abnormal condition of an organism or part, especially as a consequence of infection, inherent weakness or environmental stress, that impairs normal physiological functioning.
Drug. A substance that is used in the treatment of disease, and that in many cases has a permanent or long-term biological benefit.
Lipophilic. Soluble with fats or lipids.
Pharmaceutical ingredient. A drug.

The lipophilic cosmetic, cosmeceutical, or pharmaceutical ingredient(s) used in the invention can, by way of example and not limitation, be selected from a group consisting of emollients, skin rejuvenating agents, antioxidants, anti-inflammatory agents, antibacterial agents, antifungal agents, topical analgesic agents, local anesthetics, hormones, vitamins, nutraceutical agents, skin whitening agents, and botanical extracts.

The liposome-forming lipid(s) used in the invention can, by way of example and not limitation, be selected from a group consisting of an acyl amino acid amide (N-acyl glutamic acid diamide, for example), a siloxane-based polyamide, or from a number of polyamides marketed by Arizona Chemical under various trade names like Sylvaclear, Sylvaclear Lightwax, and Sylvaclear PA20.

The solubilizer for a liposome forming lipid component(s) can, by way of example and not limitation, be selected from a group consisting of methyl soyate alkyl benzoate esters, methyl cocate, polypropylene glycols, various vegetable oils, and other solubilizers of liposome forming lipid components.

The compositions of the invention can include a carrier(s) selected from a variety of common lipophilic emollients and that can, by way of example and not limitation, be selected from a group consisting of C12-15 alkyl benzoate, behenyl benzoate, castor oil benzoate, isopropyl myristate, isopropyl palmitate, isopropyl oleat, isopropyl stearate, methyl soyate, silicone gums, olive oil, almond oi, sesame oil, coconut oil, polypropylene glycol, propylene glycol ethers, diocytl adipate, dioctyl succinate, and dioctyl fumarate. The carrier can be incorporated before, during, or after formation of liposomes.

The lipophilic pharmaceutical, cosmetic, or cosmeceutical ingredient(s) can, by way of example and not limitation, be selected from a group consisting of vitamin E, vitamin E acetate, Tocotrienol, Progesterone, Capsaicin, Capsicum oleoresin, menthol, methyl salicylate, benzophenone-3, octyl methoxycinnamate, Benzocaine, and Lidocaine. The weight percent of the lipophilic ingredient in the liposomes of the invention is in the range of 0.01% to 55%, preferably 5% to 50%.

The following examples are given by way of illustration and not limitation of the invention.

### EXAMPLE 1

This example illustrates the use of methyl soyate to function both as a carrier for loading liposomes and as a solubilizer of liposome forming lipid components.

The following components are provided:

| Item | Component | Weight % |
|---|---|---|
| 1. | Methyl soyate (a carrier) | 55.4 |
| 2. | Propyl paraben (a preservative) | 0.2 |
| 3. | Methyl soyate (a solubilizer) | 3.0 |
| 4. | N-acyl glutamic acid diamide (a liposome- | 0.5 |
| | forming lipid component) | |
| 5. | Vitamin E acetate | 40.0 |
| 6. | Kiwi fruit seed oil | 0.1 |
| 7. | Grape seed oil | 0.1 |
| 8. | Rose hip oil | 0.1 |
| 9. | Evening primrose oil | 0.1 |
| 10. | Fragrance | 0.5 |

Step Procedure
A. Mix items 3 and 4 and heat at a temperature in the range of 90 degrees C. to 110 degrees C. to produce a solution.
B. Mix items 1, 2, 6, 7, 8, and 9 and heat to a temperature in the range of 60 degrees C. to 70 degrees C. to form a composition.
C. Admix the solution of step A with the composition of step B and begin cooling the resulting mixture to a temperature in the range of 40 degrees C. to 50 degrees C. Liposomes do not form during cooling because during cooling there is no mixing or there is only very gentle mixing.
D. Ddd item 5 to the cooled mixture of Steps C, D. Mix gently. Item 5 is rapidly absorbed into the liposomes to form a liposomal mixture. Liposomes loaded with Item 5 are spontaneously formed. The liposomes are visible to the naked eye.
E. Add item 10 to the liposomal mixture of step E and mix gently to form a final liposomal mixture.
F. F. Dispense the liposomal mixture of step F into bottles and mount a pump nozzle on each bottle.
G. G. Operate the pump nozzle on a bottle to dispense the liposomal mixture from the bottle. Liposomes in the liposomal mixture become smaller and are dispensed by the pump nozzle.

### EXAMPLE 2

This example illustrates the use of a lipophilic carrier that is different from the solubilizer of the liposome forming lipid component.

The following components are provided:

| Item | Component | Weight % |
|---|---|---|
| 1. | C12-15 alkyl benzoate (a carrier) | 90.4 |
| 2. | N-acyl glutamic acid diamide (a liposome- | 1.0 |
| | forming lipid component) | |
| 3. | Vitamin E acetate | 1.0 |
| 4. | Grape seed oil | 0.1 |
| 5. | Rose hip oil | 0.1 |
| 6. | Evening primrose oil | 0.1 |
| 7. | LiquaPar (preservative) | 0.3 |
| 8. | Methyl soyate (solubilizer) | 5.0 |
| 9. | Progesterone (a pharmaceutical lipophilic ingredient) | 1.0 |
| 10. | Fragrance | 1.0 |

Step Procedure
A. Mix items 1 and 2 and heat at a temperature in the range of 90 degrees C. to 110 degrees C. to produce a solution.
B. Mix items 3, 4, 5, 6, 7, 8, and 9 and heat to a temperature in the range of 30 degrees C. to 40 degrees C. to form a composition.
C. Cool the solution of step A to a temperature in the range of 40 degrees C. to 50 degrees. Liposomes do not form during cooling because during cooling there is no mixing or is only very gentle mixing.
D. Add the cooled solution of step C to the composition of step B and mix gently to form a mixture. Loaded liiposomes form spontaneously and are visible to the naked eye.
E. Add item 10 to the mixture of step D and mix gently. A clear gel product is formed with loaded liposomes suspended in the gel.
F. Dispense the liposomal mixture of step F into bottles and mount a pump nozzle on each bottle. On further cooling the liposome gel sets up as a clear gel with visible liposome particles.
G. Operate the pump nozzle on a bottle to dispense the liposomal mixture from the bottle. Liposomes in the liposomal mixture become smaller and are dispensed by the pump nozzle without plugging or stoppage.

### EXAMPLE 3

This example illustrates the use of an alternate liposome-forming lipid component, and illustrates the use of the same ingredient both as a carrier and as the liposome-forming lipid solubilizer.

The following components are provided:

| Item | Component | Weight % |
|---|---|---|
| 1. | C12-15 alkyl benzoate (a carrier and liposome | 35.4 |
| | forming lipid solubilizer) | |
| 2. | Syvaclear PA20 (liposome-forming lipid | 20.0 |
| | component) | |
| 3. | Vitamin A acetate (lipophilic cosmeceutical ingredient) | 35.0 |
| 4. | C12-15 Alkyl Benzoate | 10.0 |

Step Procedure
A. Mix items 1 and 2 and heat at a temperature in the range of 60 degrees C. to 70 degrees C. to produce a clear solution.
B. Cool the solution of step A to a temperature in the range of 40 degrees C. to 50 degrees C. to form liposomes. Mix during cooling. Liposomes form because the solution is mixed while it cools.
C. Mix items 3 and 4 to form a solution.
D. Add the cooled solution of step B to the solution of step C and cool to room temperature. A clear gel with liposomes loaded with vitamin E acetate is formed.
E. Dispense the gel into containers.

### EXAMPLE 4

This example illustrates the use of a mixture of vegetable oils as a carrier for a pharmaceutical ingredient.

The following components are provided:

| Item | Component | Weight % |
|---|---|---|
| 1. | Soybean oil (a carrier) | 90.4 |
| 2. | N-acyl glutamic acid diamide (a liposome- | 0.8 |
| | forming lipid component) | |
| 3. | Sesame oil | 0.5 |
| 4. | Sweet almond oil | 0.5 |
| 5. | Apricot kernel oil | 0.5 |
| 6. | Jojoba oil | 0.5 |
| 7. | Mango butter | 0.5 |
| 8. | Shea butter | 0.5 |
| 9. | Rose hip oil | 0.5 |
| 10. | Macademia oil | 0.5 |
| 11. | Evening primrose oil | 0.5 |
| 12. | Avocado oil | 0.5 |
| 13. | Progesterone (pharmaceutical ingredient) | 2.0 |
| 14. | Soybean oil | 6.0 |

Step Procedure
A. Mix items 1 and 2 and heat at a temperature in the range of 90 degrees C. to 110 degrees C. to produce a clear solution.
B. Cool the solution of step A to a temperature in the range of 40 degrees C. to 50 degrees C. Liposomes do not form during cooling because during cooling there is no mixing or is only very very gentle mixing.
C. Mix items 3 to 14 and heat to a temperature in the range of 30 degrees C. to 40 degrees C. to form a clear solution.
D. Add the cooled solution of step B to the solution of step C and mix gently to instantaneously form visible liposomes loaded with progesterone.
F. Cool the liposomal mixture of step D to room temperature to form a clear gel product with suspended loaded liposomes.
G. Dispense the clear gel/liposome product of step E into containers.

### EXAMPLE 5

This example illustrates the versatility of the liposome system in loading a variety of lipophilic skin beneficial ingredients in a single step.

The following components are provided:

| Item | Component | Weight % |
|---|---|---|
| 1. | C12-15 alkyl benzoate (a carrier) | 88.1 |
| 2. | Propyl Paraben (preservative) | 0.2 |
| 3. | N-acyl glutamic acid diamide (a liposome- | 0.5 |
| | forming lipid component) | |
| 4. | Tocotrienol (skin beneficial antioxidant) | 5.0 |
| 5. | Alpha-lipoic acid (skin beneficial antioxidant) | 0.1 |
| 6. | Coenzyme Q10 (skin beneficial antioxidant) | 0.1 |
| 7. | Vitamin K (skin beneficial vitamin) | 0.1 |
| 8. | Emu oil (skin soothing agent) | 0.1 |
| 9. | Panthenol (vitamin) | 0.1 |
| 10. | Tinoguard TT (preservative) | 0.2 |
| 11. | C12-15 alkyl benzoate (a solubilizer) | 3.0 |
| 12. | Vitamin E acetate (vitamin) | 2.0 |
| 13. | Kiwi fruit seed oil (emollient) | 0.1 |
| 14. | Grapeseed oil (emollient) | 0.1 |
| 15. | Rose hip oil (emollient) | 0.1 |
| 16. | Evening primrose oil (emollient) | 0.1 |
| 17. | Fragrance | 0.1 |

Step Procedure
A. Mix items 1 to 3 and heat at a temperature in the range of 90 degrees C. to 110 degrees C. to produce a clear solution.
B. Cool the solution of step A to a temperature in the range of 40 degrees C. to 50 degrees C. Mix the solution while it is cooling. “Empty” liposomes are formed. Empty liposomes form because during cooling there is mixing.
C. Mix items 4 to 17 and heat to a temperature in the range of 40 degrees C. to 45 degrees C. to form a clear solution.
D. Add the cooled solution of step B to the solution of step C and mix gently to produce a mixture. The addition of lipophilic ingredients to the cooled solution of step B loads the empty liposomes with organic lipophilic ingredients.
E. Cool the mixture of step D to room temperature to produce a clear gel product including loaded liposomes suspended in the clear gel.
F. Dispense the clear gel product of step E into containers.

### EXAMPLE 6

This example illustrates the use of a formulation including Coenzyme Q10. Coenzyme Q10 is a popular anti-aging ingredient that is insoluble in many solubilizers. The clear gel liposome product produced in this example is loaded with solubilized Coenzyme Q10, has enhanced skin absorption, and provides bioavailable Coenzyme Q10. This example also illustrates that various skin beneficial ingredients and vitamins can be simultaneously loaded in liposomes produced according to the invention.

The following components are provided:

| Item | Component | Weight % |
|---|---|---|
| 1. | Methyl soyate | 59.7999 |
| 2. | N-acyl glutamic acid diamide (a liposome- | 0.5 |
| | forming lipid component) | |
| 3. | Vitamin E acetate | 25.0 |
| 4. | Grape seed oil | 0.1 |
| 5. | Rose hip oil | 0.1 |
| 6. | Evening primrose oil | 0.1 |
| 7. | LiquaPar (preservative) | 0.3 |
| 8. | Methyl soyate (solubilizer) | 10.0 |
| 9. | Progesterone (a pharmaceutical lipophilic ingredient) | 1.0 |
| 10. | Kiwi fruit seed oil | 0.1 |
| 11. | Co-enzyme Q-10 | 2.5 |
| 12. | Alpha-lipoic acid | 0.0001 |
| 13. | Fragrance | 0.5 |

Step Procedure
A. Mix items 1 and 2 and heat at a temperature in the range of 90 degrees C. to 110 degrees C. to produce a clear solution.
B. Cool the solution of step A to a temperature in the range of 40 degrees C. to 50 degrees. Liposomes do not form during cooling because during cooling there is no mixing or is only very gentle mixing.
C. Mix items 4 to 13 and heat to a temperature in the range of 40 degrees C. to 45 degrees C. to form a clear solution. Liposomes do not form in the solution.
D. Add the clear solution of step C to the cooled solution of step B. Mix gently. The addition of lipophilic ingredients to the solution of step A causes loaded liposomes to form spontaneously. A clear gel with suspended liposomes results.
E. Cool the gel of step D to room temperature.
F. Dispense the liposomal mixture of step E into containers.

### EXAMPLE 7

This example illustrates that high levels of oil-soluble ingredients can be loaded in liposomes produced in accordance with the invention. In this example, the liposomes contain forty (40%) percent by weight of vitamin E acetate.

The following components are provided:

| Item | Component | Weight % |
|---|---|---|
| 1. | Methyl soyate (a carrier) | 58.0 |
| 2. | N-acyl glutamic acid diamide (a liposome- | 0.5 |
| | forming lipid component) | |
| 3. | Vitamin E acetate | 40.0 |
| 4. | Grape seed oil | 0.1 |
| 5. | Rose hip oil | 0.1 |
| 6. | Pistachio nut oil | 0.1 |
| 7. | LiquaPar (preservative) | 0.2 |
| 8. | Tetrahydrocurcumin | 0.5 |
| 9. | Fragrance | 0.5 |

Step Procedure
A. Mix items 1 and 2 and heat at a temperature in the range of 90 degrees C. to 110 degrees C. to produce a clear solution.
B. Cool the solution of step A to a temperature of 40 degrees C. to 50 degrees C. Liposomes do not form during cooling because during cooling there is no mixing or is only very very gentle mixing.
C. Mix items 3 to 9 to form a clear solution.
D. Mix the cooled solution of step B with the solution of step C. The addition of lipophilic ingredients to the cooled solution of step B causes loaded liposomes to formed spontaneously and a clear gel results in which liposomes are suspended in the gel. The solution of A is cooled because the lipophilic ingredients that are loaded into the liposomes typically are unstable at higher temperatures.
E. Cool the gel of step D to room temperature.
F. Dispense the liposomal gel of step E into containers.

Having described my invention in such terms as to enable those of skill in the art to make and practice it, and having described the presently preferred embodiments thereof,

1. A method of producing a lipophilic liposomal composition, comprising the steps of
(a) forming a liposome forming composition by admixing at an elevated temperature
(i) at least one lipophilic ingredient selected from a group consisting of pharmaceutical lipophilic ingredients, cosmetic lipophilic ingredients, and cosmeceutical lipophilic ingredients,
(ii) a liposome-forming lipid component, and
(iii) a solubliizer for said liposome-forming lipid component;
(b) cooling said liposome forming composition to form loaded liposomes.
